(19) United States
(12) Patent Application Publication
(10) Pub. No.: US 2007/0186355 A1
(43) Pub. Date: Aug. 16, 2007

(54) Method and apparatus for removing stains on articles of clothing

(75) Inventors: Michelle Lee Cancelmo, Waterford, MI (US); Kathryn Wilson-Duprey, Alexandria, VA (US)

Correspondence Address: Michael D. Wiggins, 950 Harmon, Birmingham, MI, 48009, US

(73) Assignee: Michelle Lee Cancelmo

(21) Appl. No.: 11/353,455

(22) Filed: Feb. 14, 2006

(51) Int. Cl. A01H 5/02

(52) U.S. Cl. 8/147; 15/209.1; 15/244.1

(57) ABSTRACT

A stain removing device comprises a base portion, a foam material that comprises polyurethane foam and a fastener that connects the foam material to the base portion. A handle extends from the base portion. The fastener comprises at least one of adhesives and mechanical fasteners. The fastener removably attaches the foam material to the base portion. The foam material may comprise polyester polyurethane foam.

### FIELD

The present disclosure relates to methods and apparatus for removing stains on articles of clothing.

### BACKGROUND

People commonly use deodorant to reduce body odor and/or perspiration. During the morning, people apply deodorant under their arms and then dress. When applying deodorant, the deodorant may not dry quickly. If the person applying the deodorant dresses immediately after application, the deodorant may leave a stain on clothing that comes into contact with the deodorant. Some types of deodorant tend to leave a white stain on clothing. If the contact occurs on outwardly-facing portions of the clothing, the stain is visible to others.

Removing the deodorant stain may be difficult to accomplish without removing the affected clothing and washing and/or dry-cleaning them. This approach can be inconvenient; particularly when the stain is discovered after the person leaves home. One approach for cleaning the stain includes using a damp cloth to clean the affected area. While this initially seems to correct the problem, the stain often reappears when the stained area dries.

### SUMMARY

A stain removing device comprises a base portion, a foam material that comprises polyurethane foam and a fastener that connects the foam material to the base portion.

In other features, a handle extends from the base portion. The fastener comprises at least one of adhesives and mechanical fasteners. The fastener removably attaches the foam material to the base portion.

In other features, the foam material comprises polyester polyurethane foam. The foam material has a density of approximately 1.55+/−10% lbs/ft³. The foam material has a minimum tensile strength of approximately 23.0 pounds per square inch (psi). The foam material has a minimum tear resistance of 3.0 pounds per lineal inch (pli). The foam material has a minimum compression force deflection of approximately 0.35 to 0.40 psi at 25% deflection and approximately 0.40 to 0.45 psi at 50% deflection. The foam material has an average compression force deflection of approximately 0.55 psi at 25% deflection and approximately 0.60 psi at 50% deflection.

A method for removing stains comprises providing a foam material that comprises polyurethane foam; and rubbing the foam material on a portion of clothing that includes a stain.

In other features, the method comprises attaching the foam material to a supporting base portion using at least one of adhesives and mechanical fasteners. The method comprises providing a handle that extends from the base portion. The method comprises removably attaching the foam material to the base portion.

The foam material comprises polyester polyurethane foam. The foam material has a density of approximately 1.55+/−10% lbs/ft³. The foam material has a minimum tensile strength of approximately 23.0 pounds per square inch (psi). The foam material has a minimum tear resistance of 3.0 pounds per lineal inch (pli). The foam material has a minimum compression force deflection of approximately 0.35 to 0.40 psi at 25% deflection and approximately 0.40 to 0.45 psi at 50% deflection. The foam material has an average compression force deflection of approximately 0.55 psi at 25% deflection and approximately 0.60 psi at 50% deflection.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a cross sectional view of a first exemplary stain removing device;

FIG. 2 is a top plan view of the stain removing device of FIG. 1; and

FIG. 3 is a cross sectional view of a second exemplary stain removing device;

FIG. 4 is a top plan view of the stain removing device of FIG. 3;

FIG. 5 illustrates steps of a method for reducing deodorant stains and/or other stains from clothing and other objects;

FIG. 6A is a side view of multi-layer stain removing device; and

FIG. 6B is a plan view of the multi-layer stain removing device of FIG. 6A.

### DETAILED DESCRIPTION

The following description is merely exemplary in nature and is not intended to limit the present disclosure, application, or uses. It should be understood that throughout the drawings, corresponding reference numerals indicate like or corresponding parts and features.

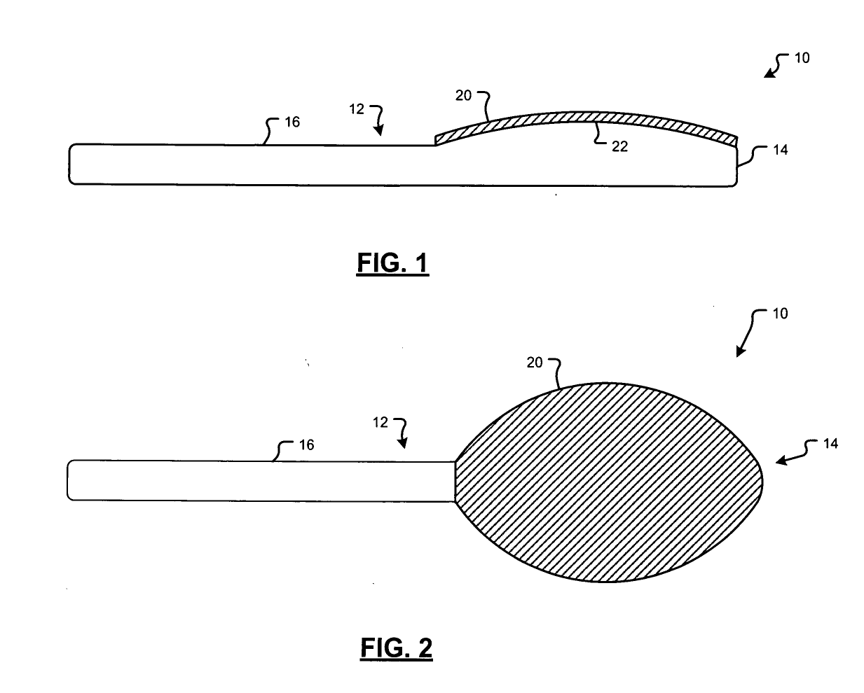

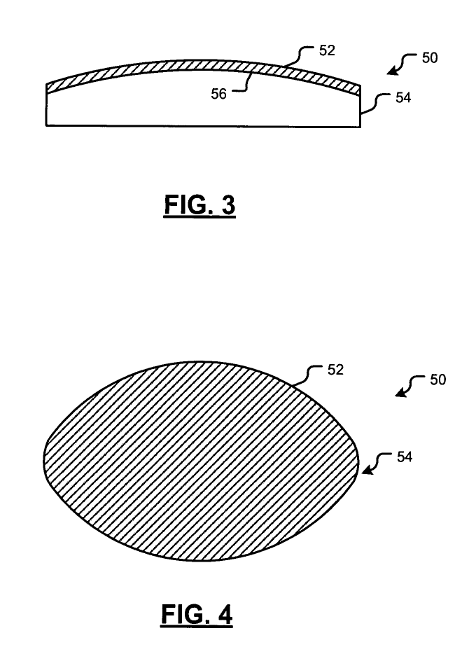

Referring now to FIGS. 1 and 2, an exemplary stain removing device 10 is shown to include a base portion 14 and a handle 16. A foam material 20 is attached to the base portion 14 using any suitable fastener 22. The fastener 22 may permanently affix the foam material 20 to the base portion 14 and/or may removably attach the foam material 20 to the base portion 14. The handle 16 may extend a variable distance from the base portion 14 depending upon a particular application. Alternately, the handle 16 can be omitted if desired as will be shown in FIGS. 3 and 4 described below. The base portion 14 may be made of any suitable material including plastic, wood, and/or other suitable materials. The base portion 14 preferably includes a material that is stiffer than the foam material 20.

Referring now to FIGS. 3 and 4, an exemplary stain removing device 50 is shown to include a base portion 54 and a foam material 52 that is attached to the base portion 54 using any suitable fastener 56. The fastener 56 may permanently affix the foam material 52 to the base portion 54 and/or may removably attach the foam material 52 to the base portion 54.

The fasteners 22 and 56 may include adhesives such as glue, epoxy, and/or other suitable adhesives and/or mechanical fasteners such as staples, Velcro® and/or other suitable mechanical fasteners. For example, the foam material 20 may be replaceable by attaching the foam material 20 to the base portion 14 using Velcro®. Further exemplary removable fasteners are disclosed in “Lotion Applicator”, U.S. Patent Publication No. 2005/0175677, published Aug. 11, 2005, which is hereby incorporated by reference in its entirety. Still other fixed and removable attachment methods are contemplated.

The foam material 20 and 52 may include polyurethane foam. The foam material 20 and 52 may include polyester polyurethane foam. In some implementations, the foam material 20 and 52 may have a density of approximately 1.55+/−10% lbs/ft³. The foam material 20 and 52 may have a minimum tensile strength of 23.0 pounds per square inch (psi) and an average tensile strength of approximately 28.0 psi. The foam material 20 and 52 may have a minimum tear resistance of 3.0 pounds per lineal inch (pli) and an average tear resistance of approximately 3.5 pli. The foam material 20 and 52 may have a minimum compression force deflection of 0.35 to 0.40 psi at 25% deflection and 0.40 to 0.45 psi at 50% deflection. The foam material 20 and 52 may have an approximate average compression force deflection of 0.55 psi at 25% deflection and 0.60 psi at 50% deflection. One or both sides of the base portion 14 and 54 may be covered with the foam material 20 and 52.

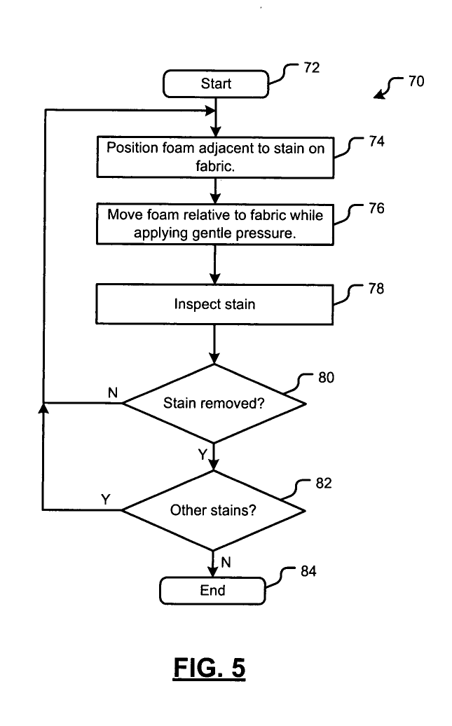

Referring now to FIG. 5, steps for removing stains from clothing are shown generally at 70. The method begins in step 72. In step 74, the foam is positioned adjacent to the stain on a fabric such as clothing. In step 76, the foam is moved relative to the fabric while applying gentle pressure. In step 78, the stain is inspected to determine whether the stain has been removed. If the stain has not been removed as determined in step 80, steps 74-78 are repeated. If the stain has been removed as determined in step 80, steps 74-80 are repeated for other stains if needed. If there are no other stains as determined in step 82, the method ends.

The above described method can be implemented with the stain removing devices described above in conjunction with FIGS. 1-4, which include a base portion, and/or without a base portion. For example, the foam material 20 and 52 may be cut into relatively small sections that can be easily attached to deodorant packaging and sold together with the deodorant.

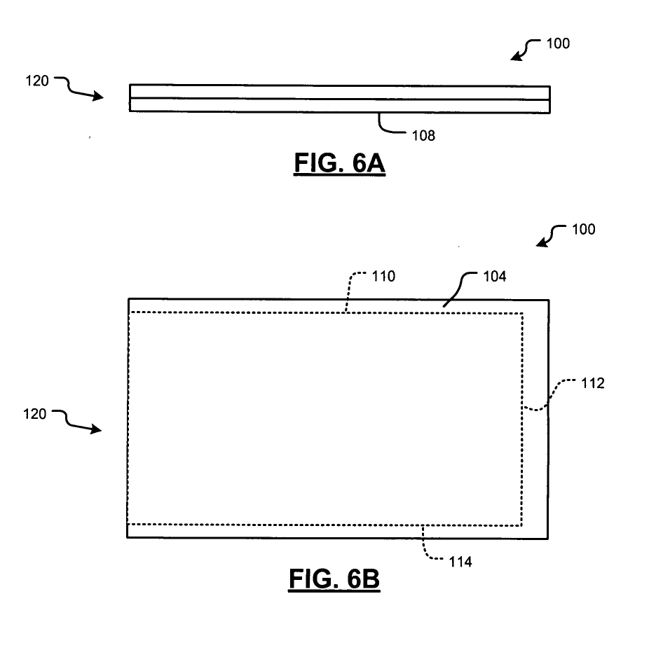

Referring now to FIGS. 6A and 6B, a multi-layer stain removing device 100 is shown. The stain removing device includes a first layer 104 and a second layer 108. An outer periphery of two or more sides of the first and second layers 104 and 108 are attached together using an attachment method indicated by dotted lines 110, 112 and 114 to define a pocket 118. The attachment method may include the use of adhesives, stitching, staples, or any other suitable method.

At least one of the first and second layers 104 and 108 includes the foam material described above. The other of the first and second layers 104 and 108 can be selected from any other suitable material such as the foam material, felt, cloth, paper or cardboard, plastic, and/or any other suitable material. In the exemplary embodiment shown in FIGS. 6A and 6B, the first and second layers 104 and 108 are attached on three sides. An opening 120 is defined on the remaining side of the first and second layers 104 and 108.

In use, a user can insert a finger, multiple fingers, a hand, etc. through the opening 120 and into the pocket 118. Then, the user rubs the foam material on the stain. The arrangement shown in FIGS. 6A and 6B allows the user to position and manipulate the foam material more easily.

As can be appreciated, the methods and apparatus described above are particularly suited for removing deodorant stains on clothing. However, this application of the methods and apparatus described herein is merely exemplary in nature and not limiting. There are other many other stain removing applications for the methods and apparatus described above.

What is claimed is:

1. A stain removing device, comprising:
a base portion;
a foam material that comprises polyurethane foam; and
a fastener that connects the foam material to the base portion.

2. The stain removing device of claim 1 further comprising a handle extending from the base portion.

3. The stain removing device of claim 1 wherein the fastener comprises at least one of adhesives and mechanical fasteners.

4. The stain removing device of claim 1 wherein the fastener removably attaches the foam material to the base portion.

5. The stain removing device of claim 1 wherein the foam material comprises polyester polyurethane foam.

6. The stain removing device of claim 1 wherein the foam material has a density of approximately 1.55+/−10% lbs/ft³.

7. The stain removing device of claim 1 wherein the foam material has at least one of:
a minimum tensile strength of approximately 23.0 pounds per square inch (psi);
a minimum tear resistance of 3.0 pounds per lineal inch (pli);
a minimum compression force deflection of approximately 0.35 to 0.40 psi at 25% deflection and approximately 0.40 to 0.45 psi at 50% deflection; and
an average compression force deflection of approximately 0.55 psi at 25% deflection and approximately 0.60 psi at 50% deflection.

8. A method for removing stains, comprising:
providing a foam material that comprises polyurethane foam; and
rubbing the foam material on a portion of clothing that includes a stain.

9. The method of claim 8 further comprising attaching the foam material to a supporting base portion using at least one of adhesives and mechanical fasteners.

10. The method of claim 9 further comprising providing a handle that extends from the base portion.

11. The method of claim 9 further comprising removably attaching the foam material to the base portion.

12. The method of claim 8 wherein the foam material comprises polyester polyurethane foam.

13. The method of claim 8 wherein the foam material has a density of approximately 1.55+/−10% lbs/ft³.

14. The method of claim 8 wherein the foam material has at least one of:
a minimum tensile strength of approximately 23.0 pounds per square inch (psi);
a minimum tear resistance of 3.0 pounds per lineal inch (pli);
a minimum compression force deflection of approximately 0.35 to 0.40 psi at 25% deflection and approximately 0.40 to 0.45 psi at 50% deflection; and
an average compression force deflection of approximately 0.55 psi at 25% deflection and approximately 0.60 psi at 50% deflection.

15. A stain removing device, comprising:
a first layer;
a second layer; and
a fastener that attaches the first layer to the second layer,
wherein at least one of the first and second layers includes a foam material that comprises polyurethane foam.

16. The stain removing device of claim 15 wherein the fastener comprises at least one of adhesive, stitching, and staples and wherein the fastener attaches at least two sides of the first and second layers.

17. The stain removing device of claim 15 wherein the foam material comprises polyester polyurethane foam.

18. The stain removing device of claim 15 wherein the foam material has a density of approximately 1.55+/−10% lbs/ft³.

19. The stain removing device of claim 15 wherein the foam material has at least one of:
a minimum tensile strength of approximately 23.0 pounds per square inch (psi);
a minimum tear resistance of 3.0 pounds per lineal inch (pli);
a minimum compression force deflection of approximately 0.35 to 0.40 psi at 25% deflection and approximately 0.40 to 0.45 psi at 50% deflection; and
an average compression force deflection of approximately 0.55 psi at 25% deflection and approximately 0.60 psi at 50% deflection.
